(19) United States
(12) Patent Application Publication
(10) Pub. No.: US 2020/0134094 A1
(43) Pub. Date: Apr. 30, 2020

(54) DROUGHT INDEX SYSTEM

(71) Applicant: NRG HOLDINGS, LLC, JERICHO, NY (US)

(72) Inventor: ELIUS ETIENNE, MAPLEWWOD, NJ (US)

(73) Assignee: NRG HOLDINGS, LLC, JERICHO, NY (US)

(21) Appl. No.: 16/171,962

(22) Filed: Oct. 26, 2018

(51) Int. Cl. G06F 17/30; G06F 3/0482; G01W 1/02; G01N 33/24; G01N 33/00; G01F 9/00

(52) U.S. Cl. CPC G06F 17/30864; G06F 3/0482; G01W 1/02; G01N 2033/245; G01N 33/0098; G01F 9/001; G01N 33/24

(57) ABSTRACT

Technologies are described for systems to generate a report. The systems may comprise a processor and a memory including instructions. The processor may be configured to receive a request for a user interface, generate interface data, send the interface data to an interface processor, and receive and save an input that includes a location and a crop. The processor may be configured to send a first query to a climate processor, receive and save climate data; send a second query to a water usage processor, receive and save water usage data; and send a third query to a water reserves processor, receive and save water reserves data. The processor may be configured to generate report data based on the input, climate data, water usage data, water reserves data, and a demand sensitive drought index algorithm, and send the report data to the interface processor to be displayed upon a display.

### BACKGROUND

Unless otherwise indicated herein, the materials described in this section are not prior art to the claims in this application and are not admitted to be prior art by inclusion in this section.

Drought may be a persistence of precipitation deficit over a period of time. Drought indices have been developed as tools for communicating drought levels. Drought indices may be based on factors such as meteorological, agricultural, hydrological, and socioeconomic variables to provide a comprehensive overview of a drought.

### SUMMARY

One embodiment of the invention is systems to generate a report. The systems may comprise a report processor and a memory including report instructions. The report instructions may include a demand sensitive drought index algorithm. The report processor may be configured to be in communication with the memory and the report processor may be in communication with an interface processor over a network. The report processor may be configured to execute the instructions to receive a request for a drought index graphical user interface from the interface processor. The report processor may be configured to generate graphical user interface data and send the graphical user interface data to the interface processor. The report processor may be configured to receive an input from the interface processor and save the input in the memory. The input may include a location and at least one of a water usage or a crop. The report processor may be configured to send a first query to a climate processor over the network. The first query may be based on the input. The report processor may be configured to receive climate data from the climate processor and save the climate data in the memory. The report processor may be configured to send a second query to a water usage processor over the network. The second query may be based on the input. The report processor may be configured to receive water usage data from the water usage processor and save the water usage data in the memory. The report processor may be configured to send a third query to a water reserves processor over the network. The third query may be based on the input. The report processor may be configured to receive water reserves data from the water reserves processor and save the water reserves data in the memory. The report processor may be configured to generate report data based on the input, the climate data, water usage data, the water reserves data, and the demand sensitive drought index algorithm. The report processor may be configured to send the report data to the interface processor to be displayed upon a display.

Another embodiment of the invention includes systems to generate a report. The systems may comprise an interface processor. The systems may comprise a memory configured to be in communication with the interface processor. The systems may comprise a display configured to be in communication with the interface processor. The interface processor may be in communication with a report processor over a network. The interface processor may be configured to send a request for a drought index graphical user interface to the report processor. The interface processor may be configured to receive graphical user interface data from the report processor. The interface processor may be configured to save the graphical user interface data to the memory. The interface processor may be configured to display the graphical user interface data on the display. The interface processor may be configured to receive an input. The input may include a location and at least one of a water usage or a crop. The interface processor may be configured to save the input in the memory. The interface processor may be configured to send the input to the report processor. The interface processor may be configured to receive report data from the report processor. The report data may be based on the input, climate data, water usage data, water reserves data, and a demand sensitive drought index algorithm. The interface processor may be configured to save the report data in the memory. The interface processor may be configured to display the report data on the display.

Another embodiment of the invention is methods to generate a report. The methods may comprise the report processor receiving an input from an application programming interface. The methods may comprise the report processor saving the input in a memory. The methods may comprise the report processor sending a first query to a climate processor over the network. The first query may be based on the input. The methods may comprise the report processor receiving climate data from the climate processor. The methods may comprise the report processor saving the climate data in the memory. The methods may comprise the report processor sending a second query to a water usage processor over the network. The second query may be based on the input. The methods may comprise the report processor receiving water usage data from the water usage processor. The methods may comprise the report processor saving the water usage data in the memory. The methods may comprise the report processor sending a third query to a water reserves processor over the network. The third query may be based on the input. The methods may comprise the report processor receiving water reserves data from the water reserves processor. The methods may comprise the report processor saving the water reserves data in the memory. The methods may comprise the report processor generating report data based on the input, the climate data, the water usage data, the water reserves data, and a demand sensitive drought index algorithm. The methods may comprise the report processor sending the report data to the application programming interface.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example system depicting an implementation of a drought index system; and

FIG. 2 illustrates a flow diagram for an example process to implement a drought index system, all arranged according to at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

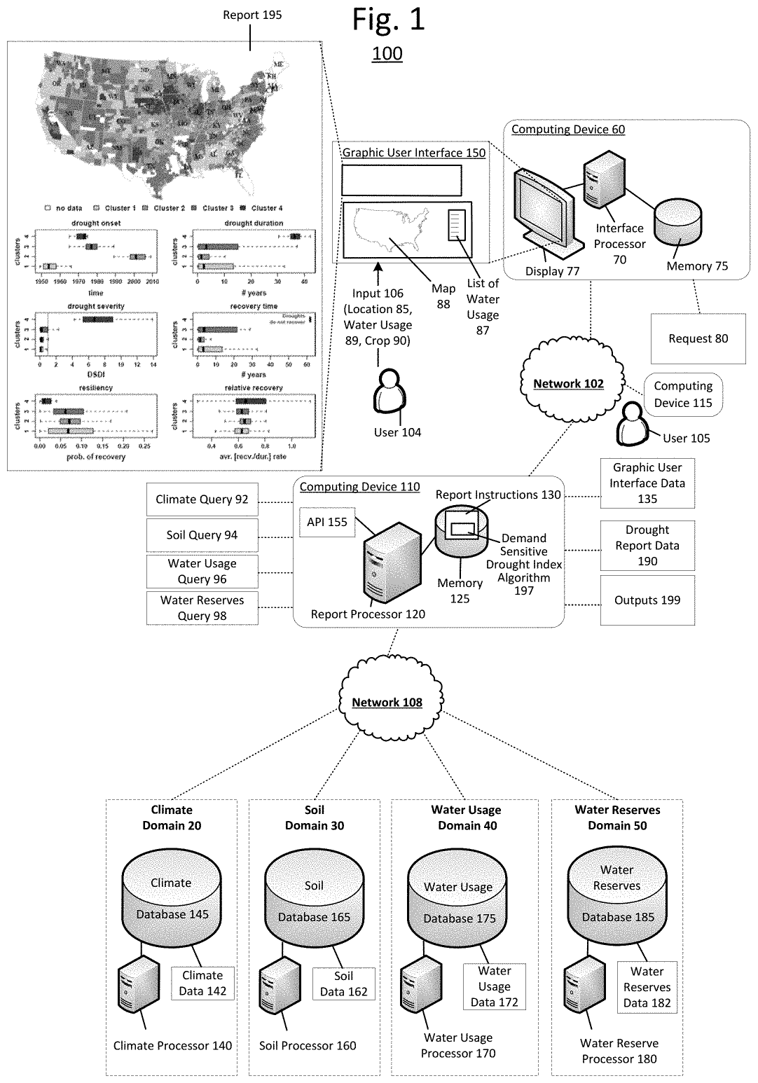

FIG. 1 illustrates an example system 100 depicting an implementation of a drought index system, arranged in accordance with at least some embodiments described herein. System 100 may include a computing device 60 configured to be in communication with a computing device 110 through a network 102. Network 102 may be the Internet, a cellular network, a personal area network, a local area network, a wide area network, etc. Computing device 60 may include a processor 70, a memory 75, and a display 77 configured to be in communication with each other. In some examples, computing device 60 may include a desktop computer, a laptop computer, etc. Computing device 110 may include a report processor 120 and a memory 125 configured to be in communication with each other. Memory 125 may include report instructions 130. Report instructions may include a demand sensitive drought index algorithm 197.

A user 104 may log into computing device 60. Processor 70 may send a request 80 to report processor 120 of computing device 110 over network 102. Request 80 may be a request for a drought index report or a drought index graphical user interface. Report processor 120 may receive request 80 and, in response, execute report instructions 130 to generate graphical user interface data 135. Graphical user interface data 135 may include data related to a graphical user interface. Graphical user interface data 135 may include data related to inputs for a drought index report.

Report processor 120 may send graphical user interface data 135 to processor 70 over network 102. Processor 70 may receive graphical user interface data 135 and save graphical user interface data to memory 75. Processor 70 may display graphical user interface 150, based on graphical user interface data 135, on display 77. Graphical user interface data 135 may include data related to a map 88 and data related to a list of water usage 87. List of water usage 87 may include items related to agricultural water usage, (e.g. a list of crops 90), industrial water usage (e.g. a list of industries such as laundries, restaurants, manufacturers, mines, etc.), or domestic water usage (a list of types of residencies, single family, apartments, condominiums, etc.). User 104 may interact with graphical user interface 150. User 104 may send an input 106 to processor 70 through graphical user interface 150. Input 106 may include a location 85 and one of a water usage 89 or a crop 90. Location 85 may be input through a selection on a map 88 displayed on graphical user interface 150. Water usage 89 or crop 90 may be input through a selection from list of water usage 87 displayed on graphical user interface 150. Processor 70 may send input 106 including location 85 and water usage 89 or crop 90 to report processor 120 over network 102.

Report processor 120 may receive input 106 including location 85 and water usage 89 or crop 90. In response to receiving input 106, report processor 120 may save location 85 and water usage 89 or crop 90 in memory 125. In response to receiving input 106, report processor 120 may execute instruction 130 to generate a climate query 92 based on location 85. Climate query 92 may include a request for climate data based at least in part on: location 85, areas related to location 85, or areas proximate to location 85. Report processor 120 may send climate query 92 to a climate processor 140 in a climate domain 20 over a network 108. Climate domain 20 may include a web site or web service of an organization or institute which gathers climate data, such as the National Oceanic and Atmospheric Administration (NOAA) or the Koninklijk Nederlands Meteorologisch Instituut (KNMI) Climate Explorer. Climate domain 20 may gather climate data through data point observations such as direct climate observations from climate stations. Climate domain 20 may gather climate data through gridded products, for example, a climate area model derived from data point observations, or a climate model derived from satellite images. Network 108 may be the Internet, a cellular network, a personal area network, a local area network, a wide area network, etc. Network 108 may be the same network as network 102 or may be a different network.

Climate processor 140 may receive climate query 92 and, in response, search climate database 145. Climate database 145 may be part of a file transfer protocol (FTP) server, an application programming interface (API), a relational database, or any other method of storing data within climate domain 20. Climate processor 140 may generate climate data 142 based on data in database 145 and climate query 92. Climate data 142 may include a text file, a comma-separated value (csv) file, an extensible markup language (XML) file, a network common data format (netCDF) file, a hierarchical data format (HDF) file, etc. Climate data 142 may include data related to climate for location 85. Climate data 142 may include data related to hourly, daily, weekly, monthly, seasonal, or yearly extremes and averages of temperature, degree days, precipitation, snowfall, snow depth, sea level pressure, relative humidity, dew point, wetbulb temperature, wind speed, solar radiation, and sky conditions. Climate data 142 may include historical climate data. Climate processor 140 may send climate data 142 to report processor 120 over network 108. Report processor 120 may receive climate data 142 and save climate data 142 in memory 125.

In response to receiving input 106, report processor 120 may execute report instructions 130 to generate a soil query 94 based on location 85. Soil query 94 may include a request for soil data based at least in part on: location 85, areas related to location 85, areas proximate to location 85, and crop 90 if a crop 90 is included in input 106. Report processor 120 may send soil query 94 to a soil processor 160 in a soil domain 30 over network 108. Soil domain 30 may include a web site or web service of an organization or institute which gathers soil data, such as the National Resources Conservation Service (NRCS), the United States Department of Agriculture (USDA), and Web Soil Survey (WSS).

Soil processor 160 may receive soil query 94 and in response search soil database 165. Soil database 165 may be part of a file transfer protocol (FTP) server, an application programming interface (API), a relational database, or any other method of storing data within soil domain 30. Soil processor 160 may generate soil data 162 based on data in database 165 and soil query 94. Soil data 162 may include data related to consistence of soil, pore size classification, rock fragment classification, soil structure, soil water data; soil texture class, percent of sand in the soil, percent of silt in the soil, and percent of clay in the soil. Soil processor 160 may send soil data 162 to report processor 120 over network 108. Report processor 120 may receive soil data 162 and save soil data 162 in memory 125.

In response to receiving input 106, report processor 120 may execute report instructions 130 to generate a water usage query 96 based on water usage 89 or crop 90. Water usage query 96 may include a request for water usage data based at least in part on location 85 and water usage 89 or crop 90. Report processor 120 may send water usage query 96 to a water usage processor 170 in a water usage domain 40 over network 108. Water usage domain 40 may include a web site or web service of an organization or institute which gathers water usage or crop data, such as the United States Department of Agriculture (USDA) and the National Agricultural Statistics Service (NASS), the Food and Agriculture Organization (FAO) statistical data on agriculture (FAOSTAT), and the United States Environmental Protection Agency (EPA).

Water usage processor 170 may receive water usage query 96 and in response search water usage database 175. Water usage database 175 may be part of a file transfer protocol (FTP) server, an application programming interface (API), a relational database, or any other method of storing data within water usage domain 40. Water usage processor 170 may generate water usage data 172 based on data in database 175 and water usage query 96. Water usage data 172 may include data related to agricultural water usage of crop 90 when input 106 includes crop 90. Water usage data 172 may include historical crop data, crop land usage, crop area planted, crop area harvested, crop price, crop stocks, crop sales, crop condition, crop soil requirements, crop evapotranspiration, crop yields, and crop growth cycles. Water usage data 172 may include data related to water usage 89 when input 106 includes water usage 89. Water usage data 172 may include data based on industrial water usage for location 85 or residential water usage for location 85. Water usage processor 170 may send water usage data 172 to report processor 120 over network 108. Report processor 120 may receive water usage data 172 and save water usage data 172 in memory 125.

In response to receiving input 106, report processor 120 may execute report instructions 130 to generate a water reserves query 98 based on location 85 and water usage 89 or crop 90. Water reserves query 98 may include a request for water reserves data based at least in part on location 85 and water usage 89 or crop 90. Report processor 120 may send water reserves query 98 to a water reserves processor 180 in a water reserves domain 50 over network 108. Water reserves domain 50 may include a web site or web service of an organization or institute which gathers water reserves data, such as the United States Geological Society (USGS) USGS National Water Information System (NWIS).

Water reserves processor 180 may receive water reserves query 98 and, in response, search water reserves database 185. Water reserves database 185 may be part of a file transfer protocol (FTP) server, an application programming interface (API), a relational database, or any other method of storing data within water reserves domain 50. Water reserves processor 180 may generate water reserves data 182 based on data in database 185 and water reserves query 98. Water reserves data 182 may include data related to surface water, ground water, precipitation, water quality, water use, streamflow, reservoirs, etc. The system can be extended to include other water reserves data as mentioned in this section. The water reserves (storages) at a location may be compared with the water requirement at that location (as determined by the drought index methodology) to assess whether an amount of water storage at the location can mitigate the drought. The water reserves and the water requirement can also be compared to estimate surpluses of water at one location that can be transferred to another location lacking water. Water reserves processor 180 may send water reserves data 182 to report processor 120 over network 108. Report processor 120 may receive water reserves data 182 and save water reserves data 182 in memory 125.

Report processor 120 may execute report instructions 130 to generate drought report data 190. Report processor 120 may generate drought report data 190 based on climate data 142, soil data 162, water usage data 172, and water reserves data 182. Report processor 120 may generate drought report data 190 based on a demand sensitive drought index (DSDI) algorithm 197 included in report instructions 130 stored in memory 125. Report processor 120 may provide area 85, water usage 89 or crop 90, climate data 142, soil data 162, water usage data 172, water reserves data 182 to demand sensitive drought index algorithm 197 to generate drought report data 190.

Demand sensitive drought index algorithm 197 may account for both water supply and demand. Demand sensitive drought index algorithm 197 may be applied to an aggregate water demand over a geographical region, or for disaggregate demand related to a specific crop or use. An output 199 of demand sensitive drought index algorithm 197 may be determined based on daily resolution of time series of supply and demand for a geographic unit j (e.g. U.S. county) as follows:

deficit_{j,t}=max(deficit_{j,t-1}+D_{j,t}−S_{j,t}, 0), where deficit_{j,t=0}=0 (1)

DICⱼ=maxₜ(deficit_{j,t;} t=1: n*365) (2)

where:
deficit_{j,t} refers to the accumulated daily deficit, D_{j,t} to total or sector wise daily water demand, S_{j,t} refers to the total daily water supply volume, for geographical location j, and day t, and n is the total number of years in the analysis.
The maximum accumulated deficit may be estimated over the n-year period without breaking the n-year period into sub-periods, and may be defined as DICⱼ (Drought Index Cumulated). DICⱼ may measure the potential impact of multiyear droughts per demand sector, or in aggregate. A corresponding normalized drought index may be:

$${DSDI}_{j} = \frac{{DIC}_{j}}{{AP}_{j}} \qquad ( 3 )$$

where APⱼ is the average annual rainfall volume (cropped area*average depth of precipitation) for county j.

Output 199 of demand sensitive drought index algorithm 197 may be derived for agriculture that may include 8 major crops (corn, soybeans, hay, wheat, barley, sorghum, rice, and cotton). The daily aggregate agricultural water demand and water supply may be determined as follows:

$$\begin{matrix}{D}_{t}^{j} = {\sum }_{m = 1}^{m = 8} \frac{{k}_{{c}_{m}}}{{β}_{m}} {ET}_{{0}_{t}}^{j} {CA}_{t}^{j , m} & ( 4 ) \\ {S}_{t}^{j} = {α}_{j} {P}_{t}^{j} {NCA}_{j} & ( 5 )\end{matrix}$$

where Dₜ^{j} is the aggregate agricultural water demand time series (t=day) for the county j, Sₜ^{j} is the water supply for the county j,
k_{cm} is the Food and Agricultural Organization of the United States (FAO) recommended crop coefficient for crop m;
ET_{O}_{ₜ}^{j,m} is the potential crop evapotranspiration determined from the Penman method,
CAₜ^{j,m} is the area planted for crop m in the county j. Crop area may change every year. Crop water demand within a year may depend on the length of the growing period for the crop and the temporal variability of the potential evapotranspiration. Since the actual water utilized on the field may be greater than the consumptive water use estimated from the empirical equations, βₘ may be a parameter to adjust for the additional losses affected from the application efficiency. βₘ=1 may be used to reflect complete efficiency. A farmer or planner may input an efficiency (based on their practices and techniques) to generate a tailored index.
Pₜ^{j} is the rainfall for day t, over the county j,
αⱼ is the factor that determines the usable fraction of rainfall by the crops over the net cropped area. αⱼ may be estimated based on the long term runoff ratio of the county. The long term runoff ratio

$$( \frac{{\overline{R}}^{j}}{{\overline{P}}^{j}} = \frac{average runoff}{average rainfall} )$$

(an index computed to understand the partitioning of rainfall into runoff and evaporation) may be related to physiographic basin features and regional climate information αⱼ may be estimated as
αⱼ as

$$1 - \frac{{\overline{R}}^{j}}{{\overline{P}}^{j}}$$

and may reflect the average fraction of rainfall that remains for the consumption of the crops after runoff. The long-term runoff ratio may be based on the hydrologic unit code level and may be aggregated to the county level.

Outputs 199 of demand sensitive drought index algorithm 197 may capture the influence of drought across years. Outputs 199 of demand sensitive drought index algorithm 197 may represent the largest cumulative deficit between renewable supply and water use over a time period. Consequently, outputs 199 of demand sensitive drought index algorithm 197 may reflect the stress associated with multi-year drought impacts at a location. The magnitude of water deficits can be interpreted as the storage required to meet the demand given a variable climate and renewable water supply. The main components of drought that may be of interest are the implications of the temporal imbalance of supply and demand at a spatial resolution consistent with decision-making. Outputs 199 of demand sensitive drought index algorithm 197 may focus on drought as defined through a temporal integration of a cumulative deficit at a daily resolution and, hence, may be examined at different levels of aggregation, e.g., seasonal, annual, or over the period of record. Although outputs 199 of demand sensitive drought index algorithm 197 are represented based on the aggregate agricultural demand, it can easily be computed as a disaggregated index specific for each crop or sector. A user may input a demand profile and obtain a customized drought index that represents the specific durations, severities and recovery times.

Drought properties such as drought onset, drought duration, drought severity, drought recovery time, and drought resiliency for a drought in a period of observation may be presented in a spatial distribution of outputs 199 of demand sensitive drought index algorithm 197. A maximum cumulative deficit may first be identified for a geographic location as a severity of a worst drought. The corresponding drought onset year, duration, recovery and resiliency may then be identified. Drought attributes may be classified using machine learning algorithms such as K-means, Decision Trees, Neural Networks, Support Vector Machines, etc. Analysis may provide objective ways to classify droughts into sub-categories depending on a multivariate dependence between the variables. Such classification may then be linked to the geographic locations to understand a spatial contiguity of droughts. For example, K-means method may be applied on the onset time and severity of a drought to find k-separations of the data based on maximum inter-cluster variations relative to centroid of each cluster and may represent K-means clustering on drought onset and severity for aggregate agricultural demand. An optimal number of clusters may be determined based on a maximum silhouette value (0.6), a measure of how cohesive each cluster is and how well the clusters are separated. A boxplot of drought attributes corresponding to each cluster may show a clear separation of clusters based on the onset of a worst drought. Demand sensitive drought index algorithm 197 may be sensitive to agricultural water demand and may show that counties that experienced drought during a time period may be counties that had agriculture prevalent during the time period of the droughts. Demand sensitive drought index algorithm 197 may complement existing drought indices such as the standardized precipitation index (SPI) or Palmer drought severity index (PDSI) by providing an impact of drought as seen from demand in a region.

Outputs 199 of demand sensitive drought index algorithm 197 may also index drought resiliency and recovery. Outputs 199 of demand sensitive drought index algorithm 197 may estimate the resiliency of a given region using two measures, the resiliency rate (i.e. the probability of recovery from a drought state) and the relative recovery (i.e. the average time it takes to completely recover from a drought compared to the duration of the drought). Outputs 199 of demand sensitive drought index algorithm 197 may be differentiated into satisfactory (S) and unsatisfactory (F) states. A satisfactory state (S) of output 199 may be identified when the cumulative deficit is either 0 or in the recedence phase (i.e. recovery time). An unsatisfactory state (F) of output 199 may be identified as the drought duration when the drought has initiated and creeping to the maximum cumulative deficit in that drought event. The transition from an unsatisfactory state (F) to a satisfactory state (S) for output 199 may be identified for a period of consideration and the county's resiliency rate may be defined as the probability of recovery from a failure state. A resiliency rate (γ) may be represented as:

$${γ}^{j} = \frac{P ( {deficit}_{j , t} ∈ F ⋂ {deficit}_{j , t + 1} ∈ S )}{P ( {deficit}_{j , t} ∈ F )} \qquad ( 6 )$$

A relative recovery (δ) may be represented as:

$${δ}^{j} = E [ \frac{{R}_{i}}{{D}_{i}} ] \qquad ( 7 )$$

and may be the expected value of the ratio of the drought recovery time to the drought duration time for a county.
Dᵢ is the drought duration
Rᵢ is the drought recovery time for each drought event i. For each county, a drought event may be defined when it has positive cumulative deficit. Within this period, the time until the maximum cumulative deficit may be the drought duration and the time to complete recedence may be the recovery time. The relative recovery (δ) may measure the rate at which a region (county in this case) will bounce back quickly from a prolonged drought. δ>1 may indicate that the drought recovery time is greater than the drought duration on average. Such regions may have slow recovery relative to the drought duration. Conversely, δ<1 may indicate that the regions have rapid recovery in relation to the drought duration.

Report processor 120 may generate drought report data 190 based on demand sensitive drought index (DSDI) algorithm 197. Report data 190 may include output 199. Report data 190 may include data related to a graphic depicting output 199, such as a map with different colors representing different values of output 199. Report processor 120 may store drought report data 190 in memory 125. Report processor 120 may send drought report data 190 to processor 70 over network 102. Processor 70 may receive drought report data 190 and, in response to receiving drought report data 190, display drought report 195 on display 77. Drought report 195 may include output 199 of demand sensitive drought index algorithm 197 or a graphic depicting output 199, such as a map with different colors representing different values of output 199. User 104 may be able to interact with drought report 195 through graphical user interface 150 to get a customized drought report 195.

In another embodiment, computing device 110 may include an application programming interface (API) 155. A user 105 may use computing device 115 or a computing system may include computing device 115 and may utilize a web service and communicate with processor 120 over network 102 through API 155. Processor 120 of computing device 110 may receive request 80 for a drought index report 195 though API 155 over network 102. Processor 120 may communicate through API 155 with computing device 115 over network 102 and may receive input 106 including location 85 and water usage 89 or crop 90 from computing device 115. In response to receiving input 106, report processor 120 may execute instruction 130 to generate a climate query 92 based on location 85. Report processor 120 may send climate query 92 to climate processor 140 in climate domain 20 over network 108. Climate processor 140 may receive climate query 92 and, in response, search climate database 145. Report processor 120 may receive climate data 142 and save climate data 142 in memory 125.

In response to receiving input 106, report processor 120 may execute report instructions 130 to generate soil query 94 based on location 85. Report processor 120 may send soil query 94 to soil processor 160 in soil domain 30 over network 108. Soil processor 160 may receive soil query 94 and in response search soil database 165. Soil processor 160 may generate soil data 162 based on data in database 165 and soil query 94. Soil processor 160 may send soil data 162 to report processor 120 over network 108. Report processor 120 may receive soil data 162 and save soil data 162 in memory 125.

In response to receiving input 106, report processor 120 may execute report instructions 130 to generate a water usage query 96 based on water usage 89 or crop 90. Report processor 120 may send water usage query 96 to water usage processor 170 in water usage domain 40 over network 108. Water usage processor 170 may receive water usage query 96 and in response search water usage database 175. Water usage processor 170 may generate water usage data 172 based on data in database 175 and water usage query 96. Water usage processor 170 may send water usage data 172 to report processor 120 over network 108. Report processor 120 may receive water usage data 172 and save water usage data 172 in memory 125.

In response to receiving input 106, report processor 120 may execute report instructions 130 to generate water reserves query 98 based on location 85 and water usage 89 or crop 90. Report processor 120 may send water reserves query 98 to a water reserves processor 180 in a water reserves domain 50 over network 108. Water reserves processor 180 may receive water reserves query 98 and, in response, search water reserves database 185. Water reserves processor 180 may generate water reserves data 182 based on data in database 185 and water reserves query 98. Water reserves processor 180 may send water reserves data 182 to report processor 120 over network 108. Report processor 120 may receive water reserves data 182 and save water reserves data 182 in memory 125.

Report processor 120 may execute report instructions 130 to generate drought report data 190. Report processor 120 may generate drought report data 190 based on climate data 142, soil data 162, water usage data 172, and water reserves data 182. Report processor 120 may generate drought report data 190 based on demand sensitive drought index (DSDI) algorithm 197 included in report instructions 130 stored in memory 125. Report processor 120 may provide area 85, water usage 89 or crop 90, climate data 142, soil data 162, water usage data 172, water reserves data 182 to demand sensitive drought index algorithm 197 to generate drought report data 190.

A system in accordance with the present disclosure may provide a user with a report that displays an index for aggregate agriculture based on two or more crops at a geographic location. A system in accordance with the present disclosure may provide a user with a report that displays an index that can be disaggregated into individual crop/demand indices. Furthermore, a report may include an index that can be derived for, or integrated with, other water use sectors such as industrial and domestic uses. A system in accordance with the present disclosure may provide a user with a report that displays an index that can assess drought impacts. A system in accordance with the present disclosure may provide a user with a report that displays an index that can break water supply and demand down into their respective components, allow a user to better understand the causes of drought frequency, duration and severity from an impact perspective.

A system in accordance with the present disclosure may provide a user with a report that displays an index that can contribute to developing more effective planning strategies for regional managers to minimize drought impacts in the current or future/projected climate and water demands. The daily integration feature of the index may make it possible for a report to examine different levels of aggregation, e.g., seasonal, annual or over a time period of record. A system in accordance with the present disclosure may provide a user with a report that displays an index that can directly inform storage requirements needed to meet the projected supply-demand imbalance at desired levels of reliability may be connected to infrastructure, planning, or water conservation needs, and may be used for the sizing of trans-basin diversions.

A system in accordance with the present disclosure may provide a user with a report that displays an index that reveals the dependence of a county on an external water source such as groundwater stores or inter-basin transfers. A system in accordance with the present disclosure may provide a user with a report that displays an index that can determine resiliency measures to understand a potential drought exposure by location. A system in accordance with the present disclosure may provide a user with a report that displays an index that can be readily accommodated for future climate scenarios to provide projected risk per demand sector, and may be integrated with a drought monitoring plan that indicates the current level of accumulated deficit or stress. A system in accordance with the present disclosure may provide a user with a report that displays an index that can determine potential impacts of climate change on supply and demand and drought impacts may be explored. A system in accordance with the present disclosure may provide a user with a report that displays an index that can determine whether conservation/efficiency improvement efforts or different ways of caching surface and groundwater storage access through infrastructure and water transfers are likely to be more effective to mitigate climate/drought impacts in a county/regional situation.

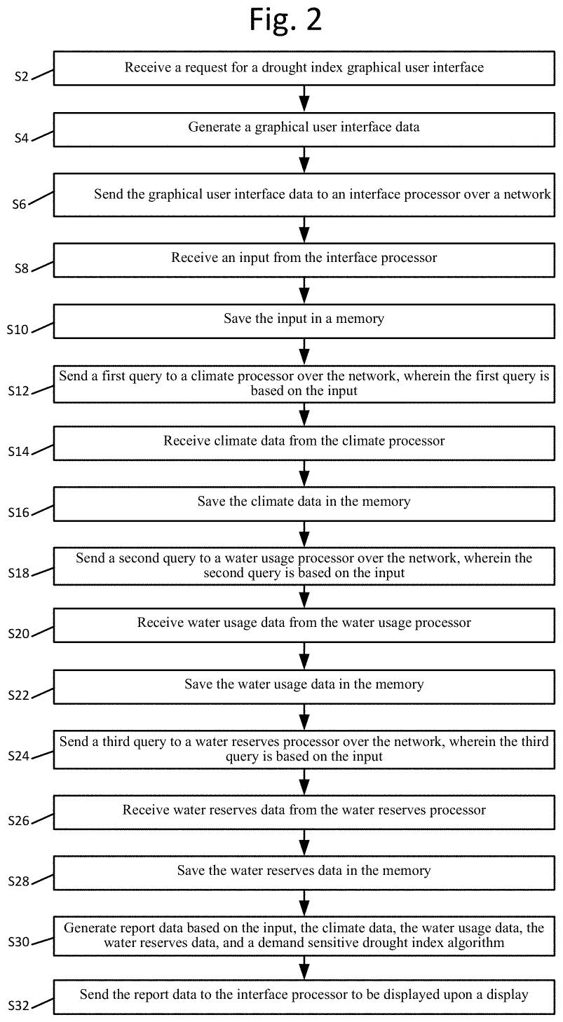

FIG. 2 illustrates a flow diagram for an example process to implement a drought index system, arranged in accordance with at least some embodiments presented herein. The process in FIG. 3 could be implemented using, for example, system 100 discussed above. An example process may include one or more operations, actions, or functions as illustrated by one or more of blocks S2, S4, S6, S8, S10, S12, S14, and/or S16. Although illustrated as discrete blocks, various blocks may be divided into additional blocks, combined into fewer blocks, or eliminated, depending on the desired implementation.

Processing may begin at block S2, “Receive a request for a drought index graphical user interface”. At block S2, a report processor may receive a request for a drought index graphical user interface form an interface processor.

Processing may continue from block S2 to block S4, “Generate a graphical user interface data”. At block S4, the report processor may to generate a graphical user interface data. The report processor may generate the graphical user interface data by executing instructions in a memory configured to be in communication with the report processor.

Processing may continue from block S4 to block S6, “Send the graphical user interface data to an interface processor over a network”. At block S6, the report processor may send the graphical user interface data to an interface processor. The interface processor may display the graphical user interface data on a display.

Processing may continue from block S6 to block S8, “Receive an input from the interface processor”. At block S8, the report processor may receive an input from the interface processor. The input may include a location and at least one of a water usage or a crop.

Processing may continue from block S8 to block S10, “Save the input in a memory”. At block S10, the report processor may save the input in a memory.

Processing may continue from block S10 to block S12, “Send a first query to a climate processor over the network, wherein the first query is based on the input”. At block S12, the report processor may send a first query to a climate processor over the network. The first query may be based on the input. The climate processor may be a processor associated with a web site or web service of an organization or institute which gathers climate data, such as the National Oceanic and Atmospheric Administration (NOAA).

Processing may continue from block S12 to block S14, “Receive climate data from the climate processor”. At block S14, the report processor may receive climate data from a climate processor. The climate data may include a text file. The climate data may include data related to climate for the location, including daily extremes of temperature, daily averages of temperature, weekly extremes of temperature, weekly averages of temperature, monthly, yearly extremes of temperature, yearly averages of temperature, dew point, wetbulb temperature, relative humidity, precipitation, snowfall, snow depth, degree days, sea level pressure, average wind speed, extreme wind speed, daily sky conditions, hourly sky conditions, solar radiation, daily precipitation, and hourly precipitation. The climate data may include historical climate data.

Processing may continue from block S14 to block S16, “Save the climate data in the memory”. At block S16, the report processor may save the climate data in the memory.

Processing may continue from block S16 to block S18, “Send a second query to a water usage processor over the network, wherein the second query is based on the input”. At block S18, the report processor may send a second query to a water usage processor over the network. The second query may be based on the input. The water usage processor may be a processor associated with a web site or web service of an organization or institute which gathers crop data, such as the United States Department of Agriculture (USDA), the National Agricultural Statistics Service (NASS), and the United States Environmental Protection Agency (EPA).

Processing may continue from block S18 to block S20, “Receive water usage data from the water usage processor”. At block S20, the report processor may receive water usage data from the water usage processor. The water usage data may include data related to agricultural water usage of the crop including historical crop data, crop land usage, crop area planted, crop area harvested, crop price, crop stocks, crop sales, crop condition, crop soil requirements, crop evapotranspiration, crop yields, and crop growth cycles. The water usage data may include data related to water usage based on industrial water usage for the location or residential water usage for the location.

Processing may continue from block S20 to block S22, “Save the water usage data in the memory”. At block S22, the report processor may save the water usage data in the memory.

Processing may continue from block S22 to block S24, “Send a third query to a water reserves processor over the network, wherein the third query is based on the input”. At block S24, the report processor may send a third query to a water reserves processor over the network. The third query may be based on the input. The water reserves processor may be a processor associated with a web site or web service of an organization or institute which gathers water reserves data, such as the United States Geological Society (USGS) USGS National Water Information System (NWIS).

Processing may continue from block S24 to block S26, “Receive water reserves data from the water reserves processor”. At block S26, the report processor may receive water reserves data from the water reserves processor. The water reserves data may include data related to surface water, ground water, precipitation, water quality, water use, streamflow, reservoirs, etc.

Processing may continue from block S26 to block S28, “Save the water reserves data in the memory”. At block S28, the report processor may save the water reserves data in the memory.

Processing may continue from block S28 to block S30, “Generate report data based on the input, the climate data, the water usage data, the water reserves data, and a demand sensitive drought index algorithm”. At block S30, the report processor may generate report data based on the input, the climate data, the water usage data, the water reserves data, and a demand sensitive drought index algorithm. The report processor may generate report data based on a demand sensitive drought index (DSDI) algorithm included in instructions stored in the memory. The report processor may provide an area, the water usage, the crop, the climate data, the water usage data, and the water reserves data to the demand sensitive drought index algorithm to generate the report data.

Processing may continue from block S30 to block S32, “Send the report data to the interface processor to be displayed upon a display”. At block S32, the report processor may send the report data to the interface processor to be displayed upon a display.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A system to generate a report, the system comprising:
a report processor;
a memory including report instructions, wherein the report instructions include a demand sensitive drought index algorithm;
wherein the report processor is configured to be in communication with the memory and the report processor is in communication with an interface processor over a network;
the report processor configured to execute the instructions to:
receive a request for a drought index graphical user interface from the interface processor;
generate graphical user interface data;
send the graphical user interface data to the interface processor; and
receive an input from the interface processor, wherein the input includes a location and at least one of a water usage or a crop;
save the input in the memory;
send a first query to a climate processor over the network, wherein the first query is based on the input;
receive climate data from the climate processor;
save the climate data in the memory;
send a second query to a water usage processor over the network, wherein the second query is based on the input;
receive water usage data from the water usage processor;
save the water usage data in the memory;
send a third query to a water reserves processor over the network, wherein the third query is based on the input;
receive water reserves data from the water reserves processor;
save the water reserves data in the memory;
generate report data based on the input, the climate data, the water usage data, the water reserves data, and the demand sensitive drought index algorithm;
send the report data to the interface processor to be displayed upon a display.

2. The system of claim 1, further comprising the report processor configured to generate an output of the demand sensitive drought index algorithm based on the climate data, the water usage data and the water reserves data and the report data includes the output of the demand sensitive drought index algorithm.

3. The system of claim 1, wherein the graphical user interface data includes data related to a map to be displayed by the interface processor.

4. The system of claim 1, wherein the graphical user interface data includes data related to water usage and includes a listing of crops to be displayed by the interface processor.

5. The system of claim 1, wherein the climate data includes at least one of hourly, daily, weekly, monthly, seasonal, or yearly extremes and averages of: temperature, degree days, precipitation, snowfall, snow depth, sea level pressure, relative humidity, dew point, wetbulb temperature, wind speed, solar radiation, and sky conditions.

6. The system of claim 1, wherein the report processor is further configured to execute the instructions to:
send a fourth query to a soil processor over the network, wherein the fourth query is based on the input;
receive soil data from the soil processor, wherein the soil data includes at least one of data related to consistence of soil, pore size classification, rock fragment classification, soil structure, soil water data; soil texture class, percent of sand in the soil, percent of silt in the soil, and percent of clay in the soil;
save the soil data in the memory; and
generate the report data based on the input, the climate data, the water usage data, the water reserves data, the demand sensitive drought index algorithm, and the soil data.

7. The system of claim 1, wherein the water usage data includes at least one of historical crop data, crop land usage, crop soil requirements, crop evapotranspiration, crop yields, and crop growth cycles.

8. The system of claim 1, wherein the water reserves data includes at least one of data related to surface water, ground water, precipitation, water quality, water use, streamflow, and reservoirs.

9. The system of claim 1, wherein the report processor is further configured to generate a drought resiliency index based on an output of the demand sensitive drought index algorithm, wherein the output of the demand sensitive drought index algorithm is based on the climate data, the water usage data and the water reserves data and the report data includes data related to the drought resiliency index.

10. The system of claim 1, wherein the report processor is further configured to generate a drought recovery index based on an output of the demand sensitive drought index algorithm, wherein the output of the demand sensitive drought index algorithm is based on the climate data, the water usage data and the water reserves data and the report data includes data related to the drought recovery index.

11. A system to generate a report, the system comprising:
an interface processor;
a memory configured to be in communication with the interface processor; and
a display configured to be in communication with the interface processor;
wherein the interface processor is in communication with a report processor over a network;
the interface processor configured to:
send a request for a drought index graphical user interface to the report processor;
receive graphical user interface data from the report processor;
save the graphical user interface data to the memory;
display the graphical user interface data on the display;
receive an input, wherein the input includes a location and at least one of a water usage or a crop;
save the input in the memory;
send the input to the report processor;
receive report data from the report processor, wherein the report data is based on the input, climate data, water usage data, water reserves data, and a demand sensitive drought index algorithm;
save the report data in the memory; and
display the report data on the display.

12. The system of claim 11, wherein the graphical user interface data includes data related to a map and the interface processor is further configured to:
display the map on the display; and
receive a selection on the map of the location.

13. The system of claim 11, wherein the graphical user interface data includes data related to water usage and includes a listing of crops, and the interface processor is further configured to:
display the listing of crops on the display; and
receive a selection of a crop from the listing of crops.

14. The system of claim 11, wherein the report data includes an output of a demand sensitive drought index algorithm.

15. A method to generate a report, the method comprising, by a report processor:
receiving an input from an application programming interface;
saving the input in a memory;
sending a first query to a climate processor over the network, wherein the first query is based on the input;
receiving climate data from the climate processor;
saving the climate data in the memory;
sending a second query to a water usage processor over the network, wherein the second query is based on the input;
receiving water usage data from the crop processor;
saving the water usage data in the memory;
sending a third query to a water reserves processor over the network, wherein the third query is based on the input;
receiving water reserves data from the water reserves processor;
saving the water reserves data in the memory;
generating report data based on the input, the climate data, the water usage data, the water reserves data, and a demand sensitive drought index algorithm; and
sending the report data to the application programming interface.

16. The method of claim 15, further comprising the report processor generating an output of the demand sensitive drought index algorithm based on the climate data, the water usage data and the water reserves data and the report data includes the output of the demand sensitive drought index algorithm.

17. The method of claim 15, further comprising the report processor generating a drought resiliency index based on an output of the demand sensitive drought index algorithm, wherein the output of the demand sensitive drought index algorithm is based on the climate data, the water usage data and the water reserves data and the report data includes data related to the drought resiliency index.

18. The method of claim 15, further comprising the report processor generating a drought recovery index based on an output of the demand sensitive drought index algorithm, wherein the output of the demand sensitive drought index algorithm is based on the climate data, the water usage data and the water reserves data and the report data includes data related to the drought recovery index.

19. The method of claim 15, wherein the input includes data related to a location and at least one of a water usage or a crop.

20. The method of claim 15, further comprising the report processor:
sending a fourth query to a soil processor over the network, wherein the fourth query is based on the input;
receiving soil data from the soil processor;
saving the soil data in the memory; and
generating the report data based on the input, the climate data, the water usage data, the water reserves data, the demand sensitive drought index algorithm, and the soil data.
